(19) United States
(12) Patent Application Publication
(10) Pub. No.: US 2004/0210159 A1
(43) Pub. Date: Oct. 21, 2004

(54) Determining a psychological state of a subject

(76) Inventor: Osman Kibar, New York, NY (US)

Correspondence Address: FISH & RICHARDSON PC, 225 FRANKLIN ST, BOSTON, MA, 02110, US

(21) Appl. No.: 10/638,239

(22) Filed: Aug. 8, 2003

Related U.S. Application Data
(60) Provisional application No. 60/462,569, filed on Apr. 15, 2003.

(51) Int. Cl. A61B 13/00; A61B 5/00; A61B 19/00

(52) U.S. Cl. 600/558; 128/898; 600/559

(57) ABSTRACT

Measurements of responses of a subject are performed automatically. The measurements include a sufficient set of measurements to complete a psychological evaluation task or to derive a complete conclusion about a cognitive state, an emotional state, or a socio-emotional state of the subject. The task is performed or the complete conclusion is derived automatically based on the measurements of responses.

This application claims the benefit of priority of U.S. provisional application serial 60/462,569, filed Apr. 15, 2003, and incorporated by reference in its entirety.

### BACKGROUND

This description relates to determining a psychological state of a subject, for example, a person or a group of people.

Knowing a subject's psychological state is useful, for example, in helping the subject to overcome psychological problems or to take advantage of psychological opportunities and to reduce risks that the subject poses to himself and to people and equipment around him.

Professional psychologists can determine the psychological state of a subject after lengthy, subjective observation, interaction, and testing.

### SUMMARY

In general, in one aspect, the invention features (a) automatically performing measurements of responses of a subject, the measurements comprising a sufficient set of measurements to complete a psychological evaluation task or to derive a complete conclusion about a cognitive state, an emotional state, or a socio-emotional state of the subject, and (b) automatically completing the task or deriving the complete conclusion based on the measurements of responses.

Implementations of the invention may include one or more of the following features: The measurements are made using electronic devices. The electronic devices include video and audio devices. Pre-stored information is automatically used to derive the complete conclusion about the cognitive state, emotional state, or socio-emotional state based on the set of measurements. An ability of the subject to carry out a function is automatically inferred, based on the complete conclusion of the cognitive state, the emotional state, or the socio-emotional state. The responses include responses to predetermined stimuli. The stimuli are automatically controlled. The stimuli are provided automatically. The stimuli comprise displayed still images or video segments. The stimuli comprise sounds. The measurements of responses include measurements of responses within a context involving subject participation or human-human interaction. The measurements of responses include measurements of responses of the subject and of other subjects involved in the subject participation or human-human interaction. The context includes the subject viewing video in a context involving subject participation or human-human interaction. The subject includes a group of humans. A conclusion is derived about the level or the quality of coordination in the group. A conclusion is derived about the level or the quality of communication in the group. A conclusion is derived about the level or the quality of cooperation in the group. A conclusion is derived on the cognitive, emotional, or socio-emotional state of a person relative to the rest of the group.

In general, in another aspect, the invention features automatically performing measurements of responses of a subject, the measurements being performed over a period of time having a pre-determined length, and automatically determining a cognitive state, an emotional state, or a socio-emotional state of the subject based on the measurements and on the length of the pre-determined period of time.

Implementations of the invention may include one or more of the following features: The measurements are also performed over a second period of time. The determination of state includes an analysis of the difference of the measurements between the period of time and the second period of time. The first period of time and the second period of time are of different scales. The different scales include at least two of: seconds, minutes, hours, days, weeks, months, or years. The measurements are also performed to determine a second state. The first state and the second state are of different time scales. The states of different time scales include at least two of emotions, moods, or temperaments. At least one measurement and at least one determined state are of different time scales.

In general, in another aspect, the invention features automatically performing measurements of responses of a subject, and automatically deriving from the measurements, a complete conclusion about a cognitive state, an emotional state, or a socio-emotional state of the subject, at least one of the measurements and the conclusions being based on a demographic characteristic of the subject.

Implementations of the invention may include one or more of the following features: The demographic characteristic includes at least one of race, gender, age, religion, culture, language, beliefs and values, education, income level, and marital status. The measurements are performed in a context that is selected to enhance a purity or intensity of the responses, the context being selected based on the demographic characteristic. The conclusion derived from the measurements is based on the demographic characteristic. An association is stored, based on the demographic characteristic, between the representations of measurements of responses and corresponding representations of the conclusion about a state.

In general, in another aspect, the invention features automatically performing measurements of responses of a subject, and automatically deriving from the measurements a complete conclusion about a cognitive state, an emotional state, or a socio-emotional state of the subject, at least one of the measurements being quantified, and the conclusion derived from the measurements being quantified.

Implementations of the invention may include one or more of the following features: An association is stored between the quantitative representations of measurements of responses and corresponding quantitative representations of the conclusion about a state. The quantitative representation includes an indicator of an intensity of the state. The accuracy or the variability of the conclusion about a state is also quantified. An association is stored between the accuracy and the variability of representations of measurements of responses and the corresponding accuracy and variability of representations of the conclusion about a state.

In general, in another aspect, the invention features, in a machine-based manner, instructing a subject to observe a performance of a multimedia work, performing the multimedia work to induce in the subject an emotional, a socio-emotional, or a cognitive state, recording responses of the subject in two different modes of expression that are associated with the state, analyzing the recording to measure the responses of the subject in the two different modes of expression, integrating the responses in the two different modes of expression, interpreting the results of the integration to provide a psychological evaluation of the subject, and presenting the evaluation results.

Implementations of the invention may include one or more of the following features: The responses include changes in the subject's face. The responses include changes in the subject's voice. The responses include changes in the subject's posture. The responses include changes in the content of a subject's speech. The responses include changes in the content of a subject's writings. The responses are also recorded before or after the performance of the multimedia work. The interpreting takes account of delays between responses in different modes of expression. The interpreting takes account of differing weights of contributions of responses in different modes of expression to determine a state. The interpreting includes comparison of the integrated responses to a norm. The evaluation results are presented as a printout to a professional or to the subject.

Other advantages and features will become apparent from the following description and from the claims.

### DESCRIPTION

FIGS. 1, 2, and 3 are block diagrams.

FIG. 4 is a flow diagram.

Man-machine interfaces (MMIs) are a broad class of technologies that either present information to a human, for example, by displaying the information on a computer screen, or provide a machine with information about a human, for example, by analyzing a facial expression or analyzing the characteristics of a voice.

A wide range of applications make use of MMIs. For example, as shown in FIG. 1, facial analysis 10 may be used to analyze a captured 12 image of a human face 14 and compare it with information about known faces 16. The identity of the human 18 may then be determined.

Some MMIs can be used to obtain information that relates to a subject's emotional state or cognitive state, that is, his mental state.

As shown in FIG. 2, by integrating two or more MMIs 20, 22 in a single application, two different kinds of information that relate to a subject's mental state can be captured 24, 26 and the captured information analyzed together 28 to produce a determination of the subject's emotional state or cognitive state 30, for example his complete emotional or cognitive state.

The MMIs include technologies 32, 34 capable of capturing the information. A wide variety of technologies may be used in various modes including (a) non-contact hardware such as auditory (e.g. voice analysis, speech recognition) or vision-based (e.g. facial expression analysis, gait analysis, head tracking, eye tracking, facial heat imaging), (b) non-contact software technologies such as artificial intelligence or content analysis software, (c) non-invasive contact hardware such as electromyagrams or galvanic skin meters, (d) invasive hardware such as brain electrodes or blood tests, and (e) contact-based software that would, for example, analyze data from the contact-based hardware.

The applications that apply the two or more MMIs may produce determinations about a wide variety of characteristics of a subject, not only cognitive or emotional states. For example, the characteristics could include symptoms (that may or may not imply a disorder), functional impairments, skills and capabilities, temperament and traits, altered thought or behavioral processes, or physiological, emotional or cognitive states and capacities. The determinations may also indicate multiple characteristics (that would imply comorbidity, or in other words, the simultaneous occurrence of more than one disorder), or undefined patterns and abnormalities.

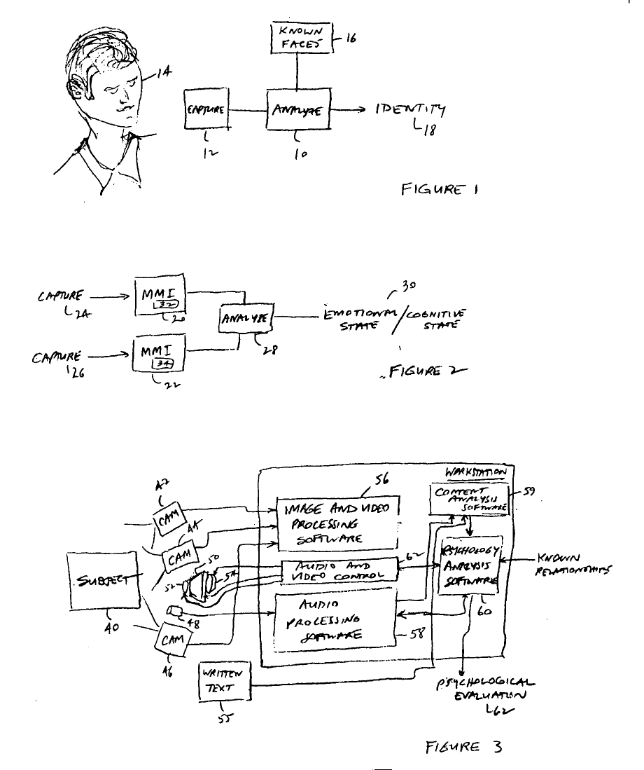

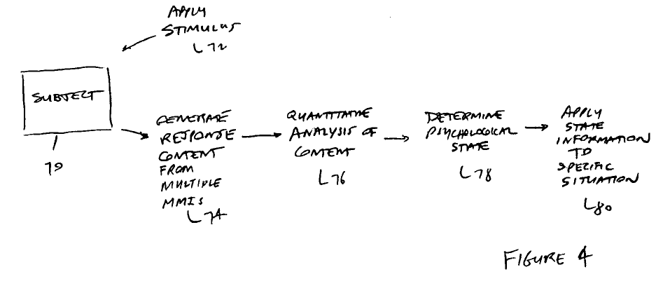

FIG. 3 shows an example of an integrated system for a clinical psychological diagnosis of a cognitive or emotional state of a subject 40.

Cognitive states are related to mental processes of knowing such as awareness, perception, reasoning, and judgment. Emotional states are related to emotions and are considered either background states, such as fatigue, wellness, or tension, or primary states such as fear, anger, or happiness. Socio-emotional states involve other people and are typically related to secondary emotions such as guilt, embarrassment, or jealousy.

In FIG. 3, to determine the cognitive or emotional state of the subject one camera 42 aimed at the subject acquires images and video sequences of the subject's head, face, eyes, and body. A second camera 44 aimed at the subject obtains images and video sequences of the subject's head, face, eyes, and body from a different angle. The two cameras 42, 44 thus provide binocular vision capable of indicating motion and features in a third dimension, e.g., depth.

A third camera 46, which is sensitive to infrared wavelengths, captures thermal images of the face of the subject. A microphone 48 detects sounds associated with speech of the subject. The three cameras and the microphone represent multiple MMIs that operate at the same time to acquire different classes of information about the subject.

An additional MMI is in the form of a digital display 50 and stereo speakers 52, 54 that provide controllable information and stimulus to the subject at the same time as the cameras and microphone are obtaining data. The information or stimulus could be images or sounds in the form of, for example, music or movies. The display and speakers can be controlled by a computer or a handheld device or by hard-wired control circuitry based on a measurement sequence that is either specified at the time of the measurement or specified at the time of the testing, by an operator or user.

The digital outputs of the three cameras 42, 44, 46 in the form of sequences of video images are communicated to image and video processing software 56. The software 56 processes the images to produce information (content) about the position, orientation, motion, and state of the head, body, face, and eyes of the subject. For example, the video processing software may include conventional routines that use the video data to track the position, motion, and orientation of the subject's head (head tracking software), the subject's body (gait analysis software), the subject's face (facial expression analysis software), and the subject's eyes (eye tracking software). The video processing software may also include conventional thermal image processing that determines thermal profiles and changes in thermal profiles of the subject's face (facial heat imaging software).

The audio output of the microphone 48 is communicated to audio processing software 58. The audio processing software includes conventional routines that determine audio characteristics of the subject's voice (voice analysis software). The audio processing software may also include conventional routines that recognize speech, and convert it to written text (speech recognition software). The output of the audio processing software is content in the form of voice characteristics and recognized speech.

The output of the speech recognition software (in 58) is delivered to the content analysis software 59. The content analysis software includes conventional routines that determine the content of the subject's spoken words, such as the coherence, completeness, and uniqueness of the thoughts and ideas that are expressed. The content analysis software 59 may also get its feed directly from written text 55 (e.g. input by the subject), rather than a speech recognition software. In other words, the content analysis software can analyze both the verbal speech and the written text of a subject.

Video and image information are delivered to a display 50 and stereo audio information is delivered to speakers 52 and 54 by audio and video control software 62. The content, amount, and timing of the video and image information and the audio information can be pre-selected to provide predetermined stimuli to the subject over a period of time in a manner that will elicit responses by the subject that are measured by the three cameras and the microphone. The selection of the stimuli may be pre-determined or may be selected by an operator of the system, for example, a psychologist based on the psychologist's judgment of stimuli that would be especially useful in eliciting responses that can be analyzed.

The audio and video control software also provides information about the timing and progress of the presented stimuli to psychology analysis software 60. The psychology analysis software can then match the stimuli with the response content being received from the image/video and audio processing and content analysis software. The psychology analysis software 60 uses the response content, the known timing of the stimuli, and known relationships between the stimuli and possible response content to provide psychological evaluations 62 of the subject. The psychological evaluations can be hypotheses or conclusions about the emotional or cognitive state of the subject.

The psychology analysis software may use a variety of known techniques, including computer science, neural network, fuzzy logic, or artificial intelligence approaches, to derive the hypotheses or conclusions. For example, the software may store rules that relate particular response content to psychological states. The software may analyze the received response content to infer categories of responses that are occurring, and then use the determined responses as the basis for triggering the stored rules.

In a specific example of use of the system of FIG. 3, the subject is instructed to watch the display and listen to the speakers (instructions may be given in written and/or verbal form via the display and speakers). The subject is then shown a movie, for example, a continuous movie or a short segment of video, or the subject is shown a still-frame, selected because they are known to induce emotional states and cognitive states such as fear, anger, happiness, confusion, frustration, or disorientation in typical subjects. The movie, segment, or frame may be (but is not required to be) interactive, inviting the subject to speak or perform actions at predetermined times. The cameras and microphone record information about the subject's responses, including his facial expressions, voice, and body movements. The information may be acquired before, during, and after the movie presentation.

The facial response content provided from the facial expression analysis software (included in the image and video processing software) is analyzed by the psychology analysis software, for example, by determining the quantitative extent of facial muscle contraction (in other words, how far the muscle have contracted), which can be indicative of sadness. The software may also determine the location and movement of specific features of the face, including the lips, nose, or eyes, and translate those determinations into corresponding psychological states using pre-existing lookup tables.

Simultaneously, from the voice characteristics provided by the voice analysis software (included in the audio processing software), the psychology analysis software may determine a reduced quantitative audibility of the subject's voice (the voice becomes softer) which may be indicative of sadness. A third analysis may determine, from the video data, a quantitative change in body posture that also indicates sadness.

Simultaneously, from the characteristics of the thoughts and ideas expressed by the subject (input directly into the computer as written text or translated into written text via the speech recognition software provided by the content analysis software), the psychology analysis software may determine an increased negativity in the subject's linguistic expressions, which may again be indicative of sadness.

The rules stored in the psychology analysis software may be invoked to determine that, when the subject has detected levels of body posture change, lowered voice audibility, muscle contraction, and negativity in the content of his speech, the subject is expressing sadness at a certain quantitative level (this could be expressed on an arbitrary scale of, say, 1 to 100 in which 100 is the saddest). The software can consider the relative intensities of the different responses and can apply corresponding weights to respective responses. The software may further conclude, from stored rules, that the subject's expressed sadness has a greater intensity or a higher frequency than in the normal population (based on data taken from a large number of subjects using similar equipment), and therefore that the subject is clinically depressed. The diagnosis would be different under the rules for different combinations and levels of responses. This conclusion (or a hypothesis) of clinical depression may be conveyed to a clinical psychologist, or other professional, thus reducing the effort required to diagnose and improving the quality of the diagnosis for the subject. In other cases, the diagnosis could be made automatically without the involvement of a professional.

The diagnosis can be presented to the professional in the form of an on-screen display or as a printout that states the clinical diagnosis, as well as the details of the reasoning used to arrive at that diagnosis, and/or the recommended actions following that diagnosis. The printout may include graphs, histograms, colors, and other visual aids.

The specific rules for operation of the psychology analysis software may be entered by one or more expert psychologists based on their knowledge of the field or based on specific tests of subjects using particular stimuli and observing the responses of the subjects. These rules for operation can also be updated in real time based on prior or current information.

Although we have referred to the content that is derived from the measurements as response content, it is possible and useful to use similar techniques in contexts in which the measurements are of parameters that have not been triggered by pre-determined and controlled stimuli but rather by conditions in the environment that are not being controlled. Thus when we refer to the subject's response, we include the situations in which the response is to stimuli that are pre-determined, response to stimuli that are not predetermined, and also to parameters of the subject that may not be considered “responses” (e.g., based on demographic factors)

An important feature of the technique in the example given above is that the response content for a given subject that is received by the psychology analysis software is analyzed quantitatively, not merely qualitatively, for the purpose of permitting automated use of the stored rules based on the quantitative results. For example, the software may quantify the subject's expression within a range of values, for example, the voice frequency or the degree of facial contractions.

Each quantification of a characteristic or parameter may be associated with statistics such as standard deviation based on empirical data. Each quantification will be compared with statistical properties of general responses such as the degree of sadness that normal subjects typically display within a timeframe and will be evaluated with respect to a psychological range such as the one between minor and major depression. As indicated earlier, the range could be an arbitrary numerical range, or a range of adjectives. Tables developed from previous experiments will provide such information, and the comparison of the fresh data with that of the tables will help to map quantitative scales of a subject's psychological or mental state.

For example, a depression scale may range from 1 to 100, where 29 and below indicates normalcy, 30 thru 50 indicates minor depression, and 51 and above indicates major depression. The scale will help to assess the degree of the subject's depression based on the response content, and the assessment will help to determine an appropriate course of treatment.

Thus, by quantifying the degree of response content for various characteristics of the subject's response, it is possible to provide repeatable, objective, and consistent results in the determination of psychological condition.

Another important feature of the techniques described here is that a complete psychological diagnosis can be made essentially automatically. Typically, to completely diagnose a subject psychologically, a professional must interview the subject and complete a mental status checklist, which includes a list of character traits such as mannerism, attitude, attention, concentration, orientation, mood, speech, insight and judgment.

The proposed technique can assess all of these characteristics completely and thereby eliminate the need for the professional's interview and/or the self-report questionnaires that are completed by the subject. For example, using techniques similar to the ones described in the earlier example, a subject's concentration level may be determined automatically by providing stimuli, measuring responses (before, during, and/or after the stimuli), quantifying the responses, and applying rules or tables to the quantified responses to reach a conclusion or hypothesis. The same approach may be applied to each of the other characteristics needed to form a complete psychological profile of a subject. Alternatively, the professional may be present to conduct the interview, but the proposed system, not the professional, may derive a conclusion from the interview's data. The professional may later use this conclusion to aid his final diagnosis. Thus, a professional may use the techniques to assist or replace him in the assessment of the cognitive, emotional, or social-emotional state of a subject.

By determining the psychological state of a subject, a subject's functional capability to carry out a given task may also be gauged.

The software could, for example, assess the cognitive state of an air traffic controller. At different times in his workday, the controller's eyelids will cover greater or lesser portions of his pupils. An increased average pupil coverage indicates an increased sleepiness. This sleepiness decreases the controller's efficiency and accuracy, making it harder for him to track items on his radar screen. The decreased efficiency and accuracy similarly imply decreased attention and energy. The software could be used to determine the psychological state or to assist a professional in determining the state. The software or the aided professional may then conclude that the controller is currently operating at less than, say, 50% alertness or capacity. The controller may then be asked to rest until his capacity returns to full, or at least to a minimum level that is predetermined to be necessary for safe operation.

Thus, the techniques permit automatic administration of all of the features of a given psychological test so that the professional can be freed from the routine of administering such a test. Professionals use certain tests to make determinations of the psychological state of a subject. Each test typically covers a variety of responses and psychological features. The test is not considered to have been completely administered unless all of the responses and all of the features have been covered and the conclusions have been drawn from all of the responses and all of the features. With the system described here, the administration of a particular test is performed automatically and completely and the result is based on all of the measured responses. Therefore, the professional can use the results with confidence that the testing was complete.

Typically, to diagnose a mental disorder clinically, a psychologist needs reports on the duration and intensity of the subject's symptoms. He also needs the results of tests he ran on the subject, as well as his own observations of the subject's behavior.

The proposed system would be able to assist or substitute for the psychologist by diagnosing a mental disorder. As an example, a desktop computer or another automated device may administer self-report tests relevant to a five-year-old subject's personality and suspected condition. In this case, the self-report tests are focused on school performance, as his suspected condition may be a learning disability. Upon completing these self-report tests and automated interviews, the subject may watch a movie that provides him with specific instructions that he should follow. After measuring and assessing the subject's responses (reactions) to the movie (where the measurements can be taken before, during, and/or after the movie), the emotional and cognitive states relevant to the five-year-old may be determined and compared with those found in current scientific literature such as DSM-IV-TR, the most recently updated version of the psychologist's manual for diagnosing mental disorders. The information provided in the manual may form the basis of rules stored in the software and used by a software engine to generate the diagnosis.

During the movie, for instance, the subject's fear (demonstrated by measurements being made by the equipment) may have increased whenever spiders appeared on screen but remained level during other frightening scenes. The system would diagnose the subject as being arachnophobic, but not subject to other phobias or anxiety disorders, based on the stored rules. More importantly, the system distinguishes the subject's mental disorder from other possible disorders and has thereby enabled a diagnostic evaluation of the subject's psychological state.

Thus, as shown in FIG. 4, in the system described above, a subject 70 receives stimuli 72 that are selected and controlled to be relevant to a psychological analysis that is to be conducted (or the stimuli may simply be environmental). Response content is generated 74 using multiple channels of information (such as video and audio). The response content is analyzed 76 to generate quantitative measurements of response characteristics. Based on the quantitative measurements, the psychological state of the subject can be automatically determined 78. The psychological state information may then be applied 80 to a specific situation to take an action or perform a step that may aid the subject, reduce risk to people around the subject, or improve the subject's performance, for example as in the example given on the air traffic controllers.

Another important aspect of the technique involves the setup of the system, which may include monitoring at different levels of states (such as symptoms, syndromes, disorders, and/or overall health), measuring rates of changes (in addition to, or instead of, absolute changes), customizing tests (according to race, gender, age, religion, culture, language, beliefs, values, education, income level, marital status, or other demographic properties), or updating equipment (including software, databases, lookup tables, etc.). The setup may also include measuring group behavior and dynamics rather than an individual or comparing individuals to groups, measuring at discrete times or over extended periods of time, or measuring in quantitative or in qualitative terms.

The system can take advantage of various time scales with respect to the measurements, the measured properties, and the results. For example, the measurements can be taken over a period that could be seconds, hours, or days. For example, a subject can be monitored for days at a time (e.g., by placing cameras and microphone recorders in his house and monitoring him during his free and private time at his home in addition to his time in the workplace). Longer observations can be done in multiple sessions or continuously. The results can then be based on measurements of varying time scales, or they can be based on the differences on the conclusions derived from shorter and longer measurements. For example, a subject's mood could be measured for an hour at the same time each day, and then his mood patterns can be derived from the variations in results from day to day.

Different time scales may also apply to the measured psychological state of the subject, for example, emotions, moods, or temperaments. Emotions are momentary affects that typically last a few seconds or minutes. Moods can last hours to days, and temperaments can last years to a lifetime.

Measurements at one time scale can be used to arrive at conclusions regarding measured properties at a different time scale. For example, a subject can be monitored for 30 minutes, and the properties of the responses he displays may be recorded and analyzed. These properties may include the severity and frequency of the responses (e.g., an intense response indicating sadness, every two minutes), as well as a specific set of expressions that he displays simultaneously or within a limited period of time (e.g., every sadness expression may be followed by a happiness expression, within the next five minutes, which may imply bipolar disorder). Based on these measurements, the system may indicate his moods and temperaments that would last much longer than 30 minutes.

The system may also measure and analyze the psychology implications of interactions of groups of subjects. For this purpose additional groups of cameras and microphones can be provided and the software can identify multiple subjects and their responses. Alternatively, the measurement and analysis can use the same system previously described and can be directed to a single subject who is interacting in a group. The integrated system can measure social interactive behavior of a subject and provide valuable information on the group dynamics of a group (e.g. level and quality of coordination, cooperation, and communication among the individuals).

For example, the subject may be engaged in a conversation with one or two other people, and the subject's behavior (and his expressions) can be analyzed to deduce his social-emotional states.

While a group of people is interacting (e.g., playing a game, performing a task, or having a conversation on a given topic), the subjects can be monitored simultaneously (e.g., by one camera recording each person's face in turn or all together at the same time and then the software identifying and analyzing each face in the image separately, or by having a dedicated camera focused on each person). Conclusions can be made on the emotional, social-emotional, and cognitive states of the whole group. For example, the group may be a cooperative group or a hostile group. It can be a group in which the workload is distributed in an efficient and optimum way. Or it can be an unproductive group which does not complete the required tasks efficiently. Each member of the group may be aware of the others, or each member may only be paying attention to himself or his own work.

Similar conclusions may be drawn by the software even without direct social interaction. For example, a movie may be shown to a subject that includes certain social interactions. His responses may be analyzed to deduce his social interactive behavior (e.g., if he were in the same situation, how would he behave?). For example, a group of characters can be shown in a certain interaction, and his eyes can be monitored to see to which subject he is paying attention to, or which character he associates himself with, or which actions in the group induce various states in the subject (such as anger or happiness).

Although specific implementations have been described above, other implementations are within the scope of the claims.

For example, the classes and examples of MMIs that are useful in psychological determinations is broad. Each of the MMIs has applications for which it is especially suitable and is appropriate for measuring specific sets of parameters of a subject. The parameters that are being measured can be completely different as between different MMIs or can be overlapping. The different MMI technologies can be used simultaneously to measure the subject or can be used sequentially depending on the specific application. The MMI technologies can be loosely categorized as hardware-based or software-based. They can also be categorized with respect to their degree of intrusiveness as no-touch, touch but non-invasive, or touch and invasive.

For example, no-touch hardware MMIs include auditory technologies, e.g., voice analysis, speech recognition, vision-based technologies, e.g., facial expression analysis (partial or full face), gait analysis (complete body or specific limb(s)), head tracking, eye tracking (iris, eyelids, pupil oscillations), infrared and heat imaging (e.g., of the face or another part of the body)

No-touch software-based technologies include artificial intelligence technologies, e.g., word selection analysis (spoken or written), and concept or content analysis (for uniqueness, completeness, and/or coherence; spoken or written).

Touch, but non-invasive, hardware-based technologies include, e.g., those that measure muscle tension (electromyagram), sweat glands and skin conductance (galvanic skin meters), heart rhythm, breathing pattern, blood pressure, skin temperature, and brain encephalagraphy.

Invasive hardware-based technologies include, e.g., electrodes placed in the brain and blood testing.

Touch, software-based technologies include, e.g., analysis software used with the touch hardware mentioned above.

A wide variety of characteristics, symptoms, or properties of a subject can be measured for use in determining the subject's cognitive state or emotional state or social-emotional state, including (a) symptoms (especially of disorders), e.g. appetite, sleep patterns, energy level, concentration, memory, (b) functional impairments, (c) skills and capacities: e.g. physical, cognitive, emotional, social-emotional, (d) temperament and traits, e.g., attention span, goal orientation, lack of distractability, curiosity, neuroticism, avoidance, impulsivity, sociopathy, self-esteem, optimism, and resilience, (e) alterations in thinking (e.g., forgetfulness as in Alzheimer's), in mood (e.g., mood swings or depression), or in behavior (e.g., attention deficit, hyperactivity), (f) physiological, emotional, or cognitive states (which do not necessarily imply a mental disorder, but could imply only a mental problem of less duration and/or intensity, or a momentary state in passing, or a pattern over time), and (g) abilities, e.g., to cope with adversity, to flourish in education or vocation or personal relationships, or to form community or spiritual or religious ties (especially in diverse cultures).

Although individual characteristics, symptoms, and properties can be measured, it is also useful to measure simultaneous occurrences (e.g., co-morbidity in disorders), and abnormalities, whether or not pre-defined. Measurements and determinations of cognitive or emotional states may also be of relative normality compared to other people.

A variety of approaches to measurement can be used.

When using a certain set of technologies to measure a given set of properties, one can also choose a specific mode of measurement.

The measurements can be made within a specific range, for example, by monitoring at the level of symptoms, syndromes, disorders, and/or overall health. Rates of decline can be measured in addition to, or instead of, absolute levels. Data can be acquired and analyzed for an individual or for an individual compared to expected group behavior. Data can be measured and analyzed at a discrete time for a given subject or over an extended period of time. If done over time, the measuring may be done continuously or at a number of discrete spaced-apart times (e.g., when following the various stages of a child's mental development).

The measurements can be done quantitatively (i.e., numbers on a scale) or in some cases qualitatively (i.e., above or below a pre-determined threshold, or as a yes/no answer based on a pre-determined definition).

A subject can be measured passively (e.g., the subject is not engaged directly or the subject is not queried about his feelings or thoughts) or actively (e.g., the subject is engaged in the measurement as in the example provided earlier). The subject may or may not be aware of being measured. The subject may be measured in the presence of an expert or while alone. The measurement result can be produced with a professional involved or in a manner of self-service usage (e.g., with no human interaction, no disclosure of identity, and no compromising of privacy).

The measurement and analysis of response content may be customized based on race, gender, age (e.g., babies, toddlers, children, adolescents, adults, and older people), religion, culture, language, beliefs and values (i.e. ethical, religious, social, family), and other demographic factors (e.g. education level, income level, marital status). This can be achieved by adjusting the stored rules that are the basis of the analysis to reflect known information about the expected responses of specific demographic groups as compared to responses of broader populations.

The tests being administered can be modified according to the subject's identity so that, for example, a given emotion can be induced in a more intense and pure state (making it easier to detect). For example, the subject can be asked to fill out a short questionnaire on his background. If he indicated that he is Asian (or if that is determined by automated analysis of his facial features) the movie clip to be shown to him could be one that uses Asian characters to which he may be more responsive.

The rules and tables stored in the psychology analysis software can be arranged for easy updating and alteration to accommodate new psychiatric or psychological research information, new diagnosis definitions and methods, new diseases, and new syndromes and symptoms. The updating could be done through a set of software tools that are exposed to users through a graphical user interface, or may be updated by delivery of new rules and tables carried on a variety of media.

Although the example provided earlier is focused on the use of the techniques in the realm of psychological assessment and diagnosis, the techniques may also be used in a wide variety of other applications and markets.

The techniques could be used in systems designed to recognize subjects and to screen subjects on the basis of psychological state. Screening could be used to identify subjects exhibiting abnormalities for closer monitoring. Such screening would not represent a diagnosis by rather a result that requires more attention to study the subject further. Screening could be done at a school of any level (e.g., kindergarten, elementary school) or any type (e.g., a regular school or a special education school) or in any place (e.g. in a classroom, or in the playground). Screening could also be done at a workplace or in a social environment. The techniques could also be used to recognize that a subject may need further study or treatment due to a mental state. For example, the screening could be performed in a healthcare setting, such as a doctor's office (e.g., with a primary care physician, a pediatrician, a psychiatrist, a mental specialist), or in a social or public setting (e.g., with a social worker).

The techniques may also be used for diagnosis (in addition to the example previously provided). In general, the techniques would be useful in psychological diagnosis to replace techniques that are currently used to manually acquire content for use in diagnosis, for example, obtaining patients' reports of intensity and duration of symptoms, accumulating signs from their mental status examination, and clinician observation of behavior including functional impairment. Automated techniques for accumulating the content will tend to eliminate or reduce over-, under-, and/or mis-diagnosis by improving objectivity and eliminating human errors and achieve better assessment of cause-correlation links, by better differentiating different disorders and behavioral abnormalities with overlapping symptoms. A subject can be passively monitored when unable (baby, language disorder) or unwilling (certain behavioral disorders, uncooperative or hostile mood) to express himself or herself.

The techniques can be used in differential diagnosis of mental states as compared to (and different from) normal developmental cycles (e.g., normal aging declines in older people, or normal cognitive and/or emotional developmental cycles for children and adolescents and even for adults).

The techniques can supplement other sources of information by acquiring the content in contexts in which a professional is not present. For example, content can be acquired in multiple settings, e.g., a home setting where a psychiatrist is not present, in which case the content would supplement reports of parents and/or family members. Thus, the techniques can provide an additional source of information (to complement information already obtained from multiple sources such as parents or teachers) to better diagnose the subject. Similarly, the techniques can supplement or replace pen and paper tests, especially to enhance information obtained on the subject's emotional and social-emotional states (as opposed to cognitive states)

The techniques are also useful in selecting and applying therapeutics with respect to psychological conditions. They can be used to monitor the progress and response of a subject during psychotherapies, including acute, continuation, and maintenance phases of therapies. Early symptoms and warning signs can be monitored on a regular basis to determine how soon and when to intervene to decrease relapse (e.g., for schizophrenia) or to prepare oneself to cope better with a relapse.

Real-world settings can be imitated during a clinical trial to reduce a gap between efficacy and effectiveness (i.e., a gap between clinical trials and real-world performance) of treatments.

The techniques can be used not only to acquire response content but also to treat conditions by operating in an interactive mode to induce a placebo effect, which may improve other treatment regimens in a cost-effective way. In cognitive-behavioral therapies, the techniques may be used as interactive feedback to enhance treatment. In addition, symptomatic responses can be monitored with respect to psychotherapies and the clinician can adjust treatment as necessary in a timely manner. Because the techniques can be applied automatically and inexpensively, they can be used for quality and outcome measures (e.g., self-monitoring or clinician-supervised monitoring over long periods of time).

In psychology or psychiatry research, the technique can be used to help differentiate cause, correlation, and consequence. For example, acquiring and analyzing content can include observing the level of a wide range of symptoms to determine which symptoms or tell-tale signs play an important role in what behavioral abnormality.

The techniques are also useful in prevention of psychological conditions. Acquiring and analyzing content from a subject before a disorder affects a subject can enable an indirect or a direct finding that the subject is susceptible.

The techniques can also be used to measure individuals who, for reasons of age, cannot express themselves well enough to permit early intervention. For example, the techniques may be used to measure competence in language of babies and/or toddlers even before they begin to talk, by measuring and analyzing responses and behavior that are known to correlate with language competence.

The techniques would also permit regular automated and low-cost mood and memory check-ups (analogous to physical check-ups) for people of all ages, especially for adults and older people. A routine series of measurements of responses that span a range of moods and a range of memory capabilities could be performed automatically on subjects. The measurements could be analyzed against stored rules to generate results that characterize the emotional or cognitive state of the subjects. These results could be provided to the subjects directly or first interpreted by a professional. The check-ups could be performed in the context of a professional's office or in a health care provider's building, or could be made available in a variety of other locations, for example, in an airport or a mall.

The techniques also enable predictions of the rates of remission, relapse, recovery, or recurrence for subjects of a given age and having a given disorder. By measuring a large number of subjects and statistically analyzing the results, it is possible to provide useful data for a variety of purposes, including insurance underwriting or clinical trials or healthcare product marketing.

Another broad area in which the techniques can be applied is psychiatry and medicine.

The techniques enable the quantification of recovery from a condition, adjustment to a condition and a level of impairment caused by a condition.

The techniques could be used in a home context to continually or repeatedly monitor subjects to determine whether treatments (especially pharmacological treatments) are being followed by the subjects, whether side effects are occurring, and whether there are any (especially long-term) side effects. The equipment to perform the measurements could be installed permanently in the home, or be portable and reusable for other subjects in other homes.

The techniques can be employed to measure and report side effects before, during, and after use of medicines. For example, the techniques could be used to measure movement disorders like body sway or postural stability associated with antipsychotic pharmacology.

The techniques could be used in the development of new drugs by measuring and analyzing the responses of subjects who are using and who are not using the new drugs. In that way, the techniques could assist in the determination of efficacy and/or safety.

The techniques are also applicable to the generation of surrogate markers during clinical trials of diseases, for example, central nervous system diseases or sleep disorders.

Statistics and detailed data on large numbers of subjects can be analyzed for use in evaluating healthcare insurance applications or in connection with pharmaceutical research and marketing (e.g., to assess compliance rates or prevalence rates)

Primary care physicians or pediatricians or other healthcare professionals, who are concerned with the overall well-being of patients, can engage in holistic monitoring of a patient. The physicians would be enabled to recognize, and not necessarily diagnose, whether a patient should be referred to a mental health specialist.

Another range of applications exists in the field of human resources and personnel evaluation.

For example, the performance of employees could be monitored by measuring their responses and behaviors with or without their knowledge. Equipment to perform the measuring could be concealed or located unobtrusively or could be located in a dedicated room. The monitoring could occur continuously or from time to time as determined by the employer, or by the employee. The data generated during successive sessions over time could be used to detect short term or long term changes in the emotional or cognitive states of the employees. Employees could be encouraged or required to take steps to alleviate problems that are identified.

Similarly, the techniques could be used to measure job satisfaction of employee in a manner and at times suggested with respect to performance monitoring.

Yet another set of applications would relate to the field of marketing. The techniques could be used as part of focus group studies with respect to new or existing products or product concepts simultaneously with the subject's direct feedback and commentary. The measurements made during the focus group studies would supplement the direct feedback to achieve higher accuracy, detail, and objectivity in the results of the study.

Military and government applications are also possible.

For example, psychological evaluations of personnel (on a routine basis or in special circumstances) by measurements of responses could be used to improve the quality and reduce the risk of assignments of personnel to tasks. They could also provide indications of the effects of trauma and other events on military personnel.

The techniques could be used in the field to monitor or measure the mental state (e.g., fatigue, sleepiness, concentration, mood) of soldiers and officers. The information could be relayed to others for further use (e.g., to a command center for coordination with other troops). The measurements could be done using equipment that is part of the soldier's clothing or helmet or part of a vehicle in which the soldier is riding. Or a portable unit could be carried into the field and used for the purpose.

In education, the techniques could be used as cognitive or emotional tutors (possibly interactive) to improve productivity of students and/or teachers.

For consumer products, the techniques could be embedded in or used to supplement fashion and/or lifestyle products. Online interactive systems (e.g., software alone, or software with some related hardware such as a camera and/or a microphone linked to the user's computer) could be created. Such systems could serve as mentors, religious guides, sources of information, personality advisors, and lifestyle (and/or dating or sex) advisors.

Applications in the financial services could include using the techniques to measure the moods of investors or traders. The measurements could be done, for example, daily or weekly or continuously and used to predict market movements and trends. New investment products could also be created based on the measured data.

In the fields of security, law enforcement, and public safety, the techniques can be used for crowd monitoring, and/or crowd control, and for monitoring or checking for illegal or dangerous activities (e.g., substance abuse, drunk driving, driving while enraged, driving while tired or sleepy). The measurements could be made openly or secretively using equipment that is apparent or equipment that is hidden.

In the field of gaming, an interactive system could be used to link a player's mental state, determined by measurement in one or more of the ways described earlier, to characters, objects, and events of the game being played.

In the case of gambling, the techniques could be used to monitor and/or derive information and statistics on behavioral trends of person(s), to screen for specific types of players and to screen for abnormal behaviors in players. Such approaches would be useful in casinos and other places in which gambling occur and also with respect to on-line gambling.

Many applications in many fields have been described above as examples. A wide variety of other applications are possible. For each application, the designer or developer would develop empirical and other evidence that would indicate which features of a subject's behavior or response should be measured, for how long, in what circumstances, and with which devices, and how the measured data for a given subject would be used to determine the emotional, the social-emotional, and/or the cognitive state of the subject.

Although particular implementations have been described above, other implementations are also within the scope of the following claims.

1. A method comprising
automatically performing measurements of responses of a subject, the measurements comprising a sufficient set of measurements to complete a psychological evaluation task or to derive a complete conclusion about a cognitive state, an emotional state, or a socio-emotional state of the subject, and
automatically completing the task or deriving the complete conclusion based on the measurements of responses.

2. The method of claim 1 in which the measurements are made using electronic devices.

3. The method of claim 2 in which the electronic devices include video and audio devices.

4. The method of claim 1 also including automatically using pre-stored information to derive the complete conclusion about the cognitive state, emotional state, or socio-emotional state based on the set of measurements.

5. The method of claim 1 also including automatically inferring an ability of the subject to carry out a function, based on the complete conclusion of the cognitive state, the emotional state, or the socio-emotional state.

6. The method of claim 1 in which the responses comprise responses to predetermined stimuli.

7. The method of claim 1 in which the stimuli are automatically controlled.

8. The method of claim 7 in which the stimuli are provided automatically.

9. The method of claim 7 in which the stimuli comprise displayed still images or video segments.

10. The method of claim 7 in which the stimuli comprise sounds.

11. The method of claim 1 in which the measurements of responses comprises measurements of responses within a context involving subject participation or human-human interaction.

12. The method of claim 11 in which the measurements of responses include measurements of responses of the subject and of other subjects involved in the subject participation or human-human interaction.

13. The method of claim 11 in which the context comprises the subject viewing video in a context involving subject participation or human-human interaction.

14. The method of claim 1 in which the subject comprises a group of humans.

15. The method of claim 14 in which a conclusion is derived about the level or the quality of coordination in the group.

16. The method of claim 14 in which a conclusion is derived about the level or the quality of the communication in the group.

17. The method of claim 14 in which a conclusion is derived about the level or the quality of the cooperation in the group.

18. The method of claim 14 in which a conclusion is derived on the cognitive, emotional, or socio-emotional state of a person relative to the rest of the group.

19. A method comprising
automatically performing measurements of responses of a subject, the measurements being performed over a period of time having a pre-determined length, and
automatically determining a cognitive state, an emotional state, or a socio-emotional state of the subject based on the measurements and on the length of the pre-determined period of time.

20. The method of claim 19 in which the measurements are also performed over a second period of time.

21. The method of claim 20 in which the determination of state includes an analysis of the difference of the measurements between the period of time and the second period of time.

22. The method of claim 20 in which the first period of time and the second period of time are of different scales.

23. The method of claim 22 in which the different scales comprise at least two of seconds, minutes, hours, days, weeks, months, or years.

24. The method of claim 19 in which the measurements are also performed to determine a second state.

25. The method of claim 24 in which the first state and the second state are of different time scales.

26. The method of claim 25 in which the states of different time scales comprise at least two of emotions, moods, or temperaments.

27. The method of claim 19 in which at least one measurement and at least one determined state are of different time scales.

28. A method comprising
automatically performing measurements of responses of a subject, and
automatically deriving from the measurements, a complete conclusion about a cognitive state, an emotional state, or a socio-emotional state of the subject,
at least one of the measurements and the conclusions being based on a demographic characteristic of the subject.

29. The method of claim 28 in which the demographic characteristic comprises at least one of race, gender, age, religion, culture, language, beliefs and values, education, income level, and marital status.

30. The method of claim 29 in which the measurements are performed in a context that is selected to enhance a purity or intensity of the responses, the context being selected based on the demographic characteristic.

31. The method of claim 29 in which the conclusion derived from the measurements is based on the demographic characteristic.

32. The method of claim 29 also including storing an association, based on the demographic characteristic, between the representations of measurements of responses and corresponding representations of the conclusion about a state.

33. A method comprising
automatically performing measurements of responses of a subject, and
automatically deriving from the measurements, a complete conclusion about a cognitive state, an emotional state, or a socio-emotional state of the subject,
at least one of the measurements being quantified, and the conclusion derived from the measurements being quantified.

34. The method of claim 33 also including storing an association between the quantitative representations of measurements of responses and corresponding quantitative representations of the conclusion about a state.

35. The method of claim 34 in which the quantitative representation comprises an indicator of an intensity of the state.

36. The method of claim 33 in which the accuracy or the variability of the conclusion about a state also being quantified.

37. The method of claim 36 also including storing an association between the accuracy and the variability of representations of measurements of responses and the corresponding accuracy and variability of representations of the conclusion about a state.

38. A machine-based method comprising
instructing a subject to observe a performance of a multimedia work,
performing the multimedia work to induce in the subject an emotional, a socio-emotional, or a cognitive state,
recording responses of the subject in two different modes of expression that are associated with the state,
analyzing the recording to measure the responses of the subject in the two different modes of expression,
integrating the responses in the two different modes of expression,
interpreting the results of the integration to provide a psychological evaluation of the subject, and
presenting the evaluation results.

39. The method of claim 38 in which the responses comprise changes in the subject's face.

40. The method of claim 38 in which the responses comprise changes in the subject's voice.

41. The method of claim 38 in which the responses comprise changes in the subject's posture.

42. The method of claim 38 in which the responses comprise changes in the content of a subject's speech.

43. The method of claim 38 in which the responses comprise changes in the content of a subject's writings.

44. The method of claim 38 in which the responses are also recorded before or after the performance of the multimedia work.

45. The method of claim 38 in which the interpreting takes account of delays between responses in different modes of expression.

46. The method of claim 38 in which the interpreting takes account of differing weights of contributions of responses in different modes of expression to determine a state.

47. The method of claim 38 in which interpreting includes comparison of the integrated responses to a norm.

48. The method of claim 38 in which the evaluation results are presented as a printout to a professional or to the subject.
